(19) United States
(12) Patent Application Publication
(10) Pub. No.: US 2026/0241138 A1
(43) Pub. Date: Aug. 20, 2026

(54) APPARATUS AND RELATED METHODS FOR FORMING A CUSTOM-LENGTH CATHETER

(71) Applicant: BARD PERIPHERAL VASCULAR, INC., Franklin Lakes, NJ (US)

(72) Inventors: Samuel SHREVE, Scottsdale, AZ (US); Gabriella ROMANO, Phoenix, AZ (US); Summer FORD, Queen Creek, AZ (US); Hunter KEARNEY, Scottsdale, AZ (US); Alex PALMER, Gilbert, AZ (US)

(21) Appl. No.: 19/162,033

(22) PCT Filed: Mar. 22, 2023
(86) PCT No.: PCT/US2023/015880

§ 371 Date: Sep. 3, 2025

(51) Int. Cl. A61M 25/00

(52) U.S. Cl. CPC A61M 25/0014; A61M 25/0026; A61M 25/0097; A61M 2025/0037

(57) ABSTRACT

A method is disclosed for manufacturing a custom length catheter including a hub from a shaft including an outer tube covering an inner tube. The method includes removing a portion of the outer tube to uncover a portion of the inner tube, and connecting the outer tube or the inner tube to the hub. Related apparatus and methods are also disclosed.

### BACKGROUND

Many catheters and, in particular, those designed for endovascular use to treat peripheral artery disease, are manufactured and distributed in a variety of lengths. These differing catheter lengths are designed to accommodate use at different treatment locations within a body lumen, such as a blood vessel. For instance, a clinician might use a balloon catheter of one length (e.g., approximately 75-80 centimeters) for antegrade femoral access to treat a lesion in the superficial femoral artery. In another situation, a clinician might employ a longer balloon catheter (e.g., approximately 150-200 centimeters) to reach a “below the knee” lesion from a radial access location.

Supplying catheters in a variety of lengths has certain downsides. For one, this practice increases the number of individual catheters that must be manufactured. As another, it may require that such catheters be made readily available for use in performing different procedures. An increase in cost and reduction in efficiency results.

Accordingly, a need is identified for a catheter (and in particular one having an expandable element, such as an inflatable balloon) that may be readily adapted for use in gaining access to different treatment locations within a body lumen. This adaptability would avoid the need for manufacturing catheters of varying lengths for use in performing procedures. Likewise, it would avoid the need for the clinician to have ready access to such different lengths of catheters in order to accommodate the needs of a particular patient intervention. A reduction in complexity and expense, including in terms of inventory management and cost, could also thus be realized by providing such a catheter that may be provided “just in time” by the clinician for use in performing a particular medical procedure.

### SUMMARY

An object of the disclosure is to provide a catheter (and in particular one having an expandable element, such as an inflatable balloon) that may be readily adapted for use in gaining access to different treatment locations within a body lumen. This adaptability would avoid the need for manufacturing catheters of varying lengths for use in performing procedures, as well as the need for the clinician to have ready access to such different lengths of catheters in order to accommodate the needs of a particular patient intervention. The reduction in complexity and expense realized by providing such a catheter that may be provided “just in time” by the clinician for use in performing a particular medical procedure.

According to one aspect of the disclosure, a method is provided for manufacturing a custom length catheter including a hub from a shaft including an outer tube covering an inner tube. The method includes removing a portion of the outer tube to uncover a portion of the inner tube. The method further includes connecting the outer tube or the inner tube to the hub.

In one example, the method further includes the step of cutting the outer tube prior to the removing step. Prior to cutting the outer tube, a guard may be provided for guarding the inner tube. The step of providing the guard may involve inserting the guard into an annulus between the outer tube and the inner tube, and forming a non-contact coupling between the guard and a cutter external to the outer tube. Alternatively, the step of providing the guard may involve inserting the guard into an annulus between the outer tube and the inner tube to a distance corresponding to a marking on the outer tube indicating a length of a remaining portion of the outer tube following the cutting and removal steps.

The connecting step may comprise connecting the outer tube to a first connector of the hub. The connecting step may further comprise connecting the inner tube to a second connector of the hub. This may involve pushing the outer or inner tube through the first or second connector, respectively. Either before or after the connecting step, a portion of the inner tube may be removed, such as by cutting, which may also be done for the outer tube prior to the connecting step with the first connector.

In some embodiments, the catheter inner tube may comprise a first inner tube, and the catheter may further include a second inner tube removably attached to the first inner tube. In such case, the method may include the steps of detaching the first and second inner tubes, and connecting at least the first inner tube to the hub. The method may further include connecting the second inner tube to the hub or to another hub. According to another aspect of the disclosure, a method for manufacturing a custom length catheter from a shaft including first and second lumens is provided. The method comprises connecting the shaft to a hub including a first connector for insertion into either the first lumen or the second lumen.

In one embodiment, the hub includes a second connector, and the method comprises inserting the second connector into the other of the first lumen or the second lumen. The method may further include the step of aligning a first guide on the hub with a second guide on the shaft to ensure connection in the correct orientation so that the first connector is connected to either the first lumen or the second lumen. Additionally, the method may comprise the step of aligning a first guide on the hub with a second guide on the shaft to ensure that the first connector is connected to the first lumen and the second connector is connected to the second lumen.

This disclosure also pertains to apparatuses for forming a custom length catheter for performing a medical procedure, the catheter having a shaft including an inner tube and an outer tube. In one aspect, the apparatus comprises a cutter adapted for cutting the outer tube and a removable guard for guarding the inner tube to protect against cutting by the cutter.

In one embodiment, the cutter is adapted for forming a non-contact coupling with the guard. This may involve providing a magnet external to the outer tube for forming a magnetic coupling with the guard. The cutter may include at least one blade for cutting the outer tube.

A further aspect of the disclosure pertains to an apparatus for forming a catheter for performing a medical procedure. The apparatus includes a catheter shaft including an outer tube and an inner tube arranged in a coaxial configuration. A hub includes at least one connector for irremovably connecting the inner tube or the outer tube to the hub. In one embodiment, the at least one connector comprises a connector captured within a passage of the hub. The at least one connector may comprise a first connector for connecting to a proximal portion of the inner tube within the hub and further including a second connector for connecting to the outer tube.

A further aspect of the disclosure pertains to an apparatus for forming a catheter for performing a medical procedure. The apparatus includes a catheter shaft including first and second lumens. A hub includes a first connector for insertion into the first lumen and a second connector for insertion into the second lumen.

In one embodiment, the first and second lumens have different cross-sectional shapes, which helps to ensure that the first connector is connected to the first lumen and the second connector is connected to the second lumen when the catheter shaft is associated with the hub. Additionally or alternatively, the apparatus may further include a first guide on the hub and a second guide on the catheter. The first and second guides are arranged to ensure that the first connector is connected to the first lumen and the second connector is connected to the second lumen when the catheter shaft is associated with the hub. The guides alone may be used, in which case the first and second lumens may each have a cross-sectional shape that is substantially the same.

### BRIEF DESCRIPTION OF THE DRAWINGS

The above and further advantages of the disclosure may be better understood by referring to the following description in conjunction with the accompanying drawings in which:

FIG. 1 schematically illustrates a first embodiment of a catheter according to the present disclosure;

FIG. 1A is a cross-sectional view taken along line 1A-1A of FIG. 1;

FIGS. 2, 3, and 4 are progressive views illustrating forming of a custom cut-to-length catheter from the catheter according to FIG. 1;

FIG. 5 schematically illustrates a second embodiment of a catheter according to the disclosure;

FIGS. 6, 7, and 8 are progressive views illustrating forming of a custom cut-to-length catheter from the catheter according to FIG. 5;

FIG. 9 schematically illustrates a third embodiment of a catheter according to the disclosure;

FIGS. 9A and 9B are cross-sectional views taken along line 9A-9A or 98-9B to show different side-by-side lumen options for the catheter of FIG. 9;

FIG. 9C schematically illustrates a variety of connectors for connecting a custom cut-to-length catheter to a hub;

FIG. 10 schematically illustrates a fourth embodiment of a catheter according to the disclosure;

FIG. 10A is a cross-sectional view taken along line 10A-10A of FIG. 10;

FIG. 10B is an enlarged view of a cutter according to the fourth embodiment of FIG. 10;

FIG. 10C is a cross-sectional view taken along line 10C-10C of FIG. 10;

FIGS. 11, 12, and 13 are progressive views illustrating forming of a custom cut-to-length catheter from the catheter according to FIG. 10; and

FIGS. 12A and 12B illustrate an example of a hub for use in connection with a custom cut-to-length catheter.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the present invention.

The principles and operation of the apparatus and methods of the disclosure may be better understood with reference to the drawings and accompanying descriptions. This disclosure is not limited in its application to the details of construction and the arrangement of the components set forth in the following description or illustrated in the drawings. The apparatus and methods are capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

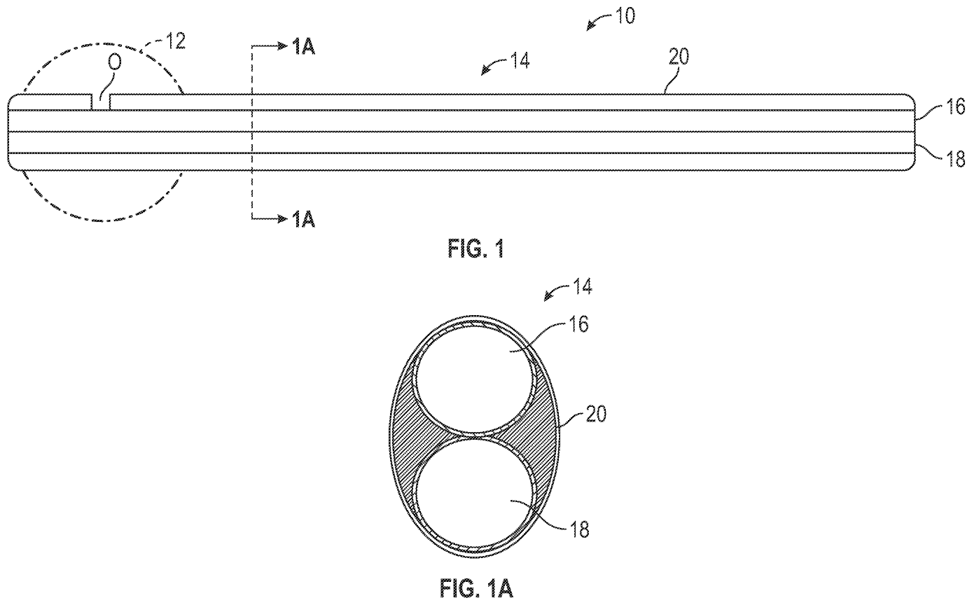

With reference to FIG. 1, a catheter 10 adapted to be cut-to-length for use in connection with a medical procedure is shown. In one exemplary use, the catheter 10 is adapted for performing percutaneous transluminal angioplasty, and thus is attached to an expandable element, such as an inflatable balloon 12 (shown in phantom line depiction). Despite this depiction, this disclosure is not limited to any particular application of the catheter in terms of use in connection with a medical procedure involving a body lumen.

The catheter 10 comprises a shaft 14, which includes one or more inner lumens. The inner lumens may be formed by one or more tubes 16, 18 at least partially surrounded and covered by an outer tubular sleeve 20. As can be appreciated from FIG. 1A, the tubes 16, 18 may be provided in a side-by-side arrangement, but could instead be co-axially arranged as outlined further in the following description. The catheter 10 as shown in FIG. 1 may be considered as a precursor for forming a custom cut-to length catheter.

The shaft 14, or sleeve 20 in particular, and thus may have an initial or nominal length L1, and as a result of cutting away at least part of the sleeve 20, may have a shorter length L2, as shown in FIG. 3. For instance, when the desired shorter length L2 comprises 75, 100, and 150 centimeters, then the nominal length L1 of the shaft 14 including the un-cut sleeve 20 is at least 150 centimeters (and could also be greater). The nominal length L1 of the full sleeve 20 is thus oversized relative a final, shorter length (see, for example, shaft 14a with sleeve portion 20b in FIG. 3) that may be formed from it, such as by cutting to achieve the desired length.

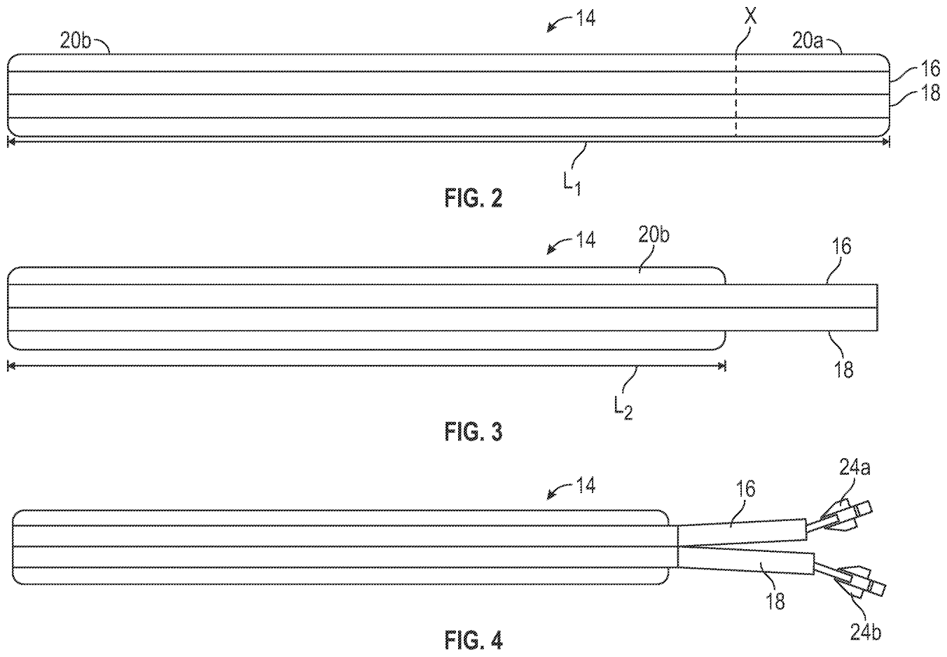

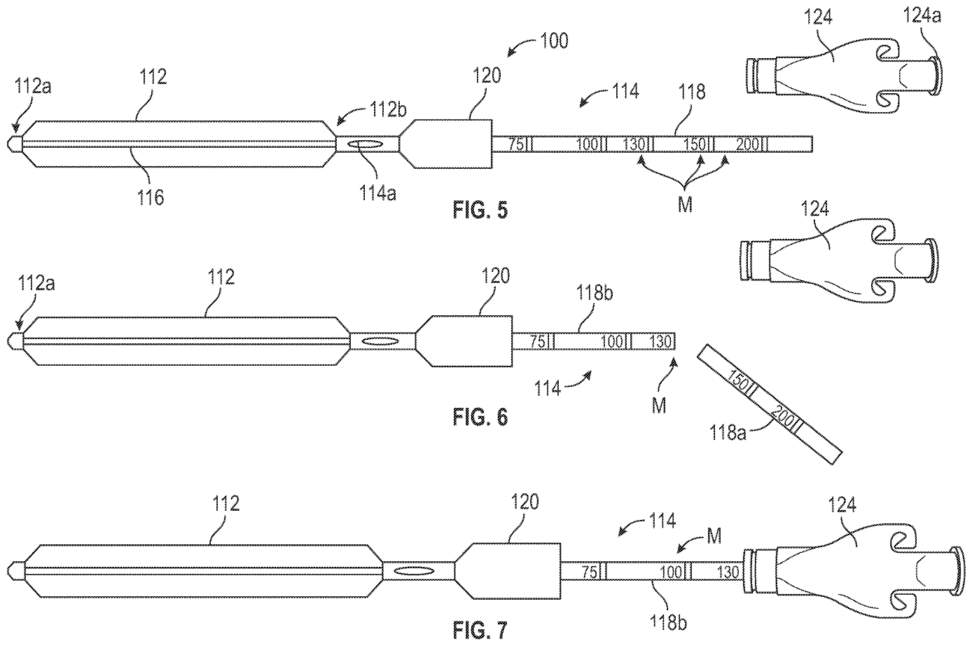

Specifically, with reference to FIG. 2, a particular length of the shaft 14 (such as length L2 or a different length depending on the nature of the intervention being performed) may be identified on the sleeve 20. This is indicated in FIG. 2 by the dashed line X, which may be imaginary or may be formed in or marked on the sleeve 20, such as by the user. This particular length L2 may correspond to one or more visually perceptible markings provided on the shaft 14 and, in particular, the sleeve 20 (see, e.g., FIG. 5). The line X may also correspond to or include a frangible portion to facilitate dividing the sleeve 20 into portions.

In any case, once the desired location is identified, the sleeve 20 may be cut or severed along line X. As a result, a portion 20a of the sleeve 20 may be removed from the shaft 14, as FIG. 3 illustrates. This removal partially exposes or uncovers the inner tubes 16, 18, which in this case are arranged side-by-side and at least partially attached to each other, as noted. The remainders of the tubes 16, 18 remain partially covered by a remaining portion 20b of the sleeve 20, and thus remain captured and incapable of fully separating from each other (at least without further removal of portion(s) of sleeve).

The tubes 16, 18 may be attached or bonded to each other, including along the now-exposed portion as a result of the partial removal of sleeve 20. This connection may be as a result of an adhesive used during the fabrication process or as a result of other manufacturing techniques (such as extrusion). In such case, the tubes 16, 18 may then be separated, such as by being peeled apart, as shown in FIG. 4. This separation occurs at least up to the point at which the tubes 16, 18 remain covered by the remaining portion 20b of sleeve 20.

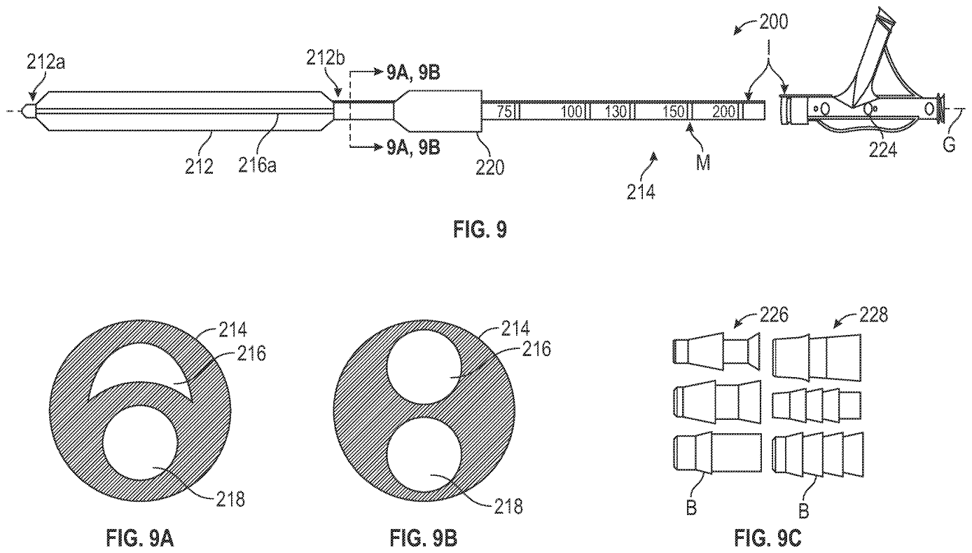

The separated tubes 16, 18 may then be associated with one or more hubs, which in the illustrated version comprise a first hub 24a for tube 16 and a second hub 24b for tube 18. A single hub (not shown, but see hub 224 in FIG. 9) could instead be used for connecting to both tubes 16, 18. As a result, a cut-to-length, custom catheter 10 may be formed in an easy and efficient manner, and may avoid the need for manufacturing a variety of catheter lengths and maintaining such on hand.

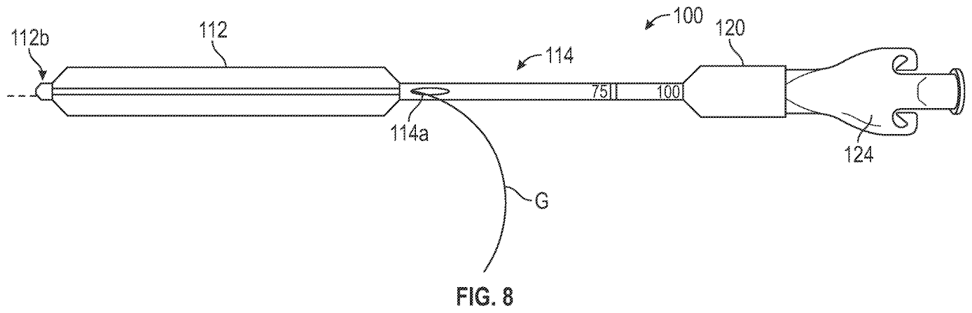

The hub or hubs 24a, 24b may be adapted for various purposes. For example, hubs 24a, 24b may be adapted for receiving a material and introducing it to one or both of the tubes 16, 18. The material may comprise, for example, an implement, such as a guidewire (see guidewire G in FIG. 8) for guiding the catheter 10 in the endovascular space or other structure for performing a medical procedure. Alternatively or additionally, the material may comprise a fluid, such as a liquid (e.g., saline) for inflating the balloon 12 via an opening or port or opening O in the shaft 14 (or possibly through the end of the corresponding tube if it does not enter the interior compartment of the balloon, as shown in FIG. 1).

The hubs 24a, 24b may also be adapted to connect to the tubes 16, 18 in a push-fit manner. This type of connection avoids the need for using a tool, and may also avoid the need for adhesives or other forms of bonding. As outlined further in the description that follows, the connection may be established in an irremovable manner, meaning the hub 24a or 24b cannot be separated from the respective tube 16, 18 without causing damage to one or both of these parts.

In the case where the hubs 24a, 24b serve different functions in terms of introducing material to the catheter 10, the tubes 16, 18 may differ in some respect to ensure that the proper pairing is made in forming the desired connection. For example, each tube 16, 18 may each be provided with a different indicia, such as for example an alphanumeric indicator, coloration, or other physical feature to allow for differentiation.

Alternatively or additionally, each tube 16, 18 may have a different shape, such as in cross-section. Corresponding structures provided in the hub 24a or 24b, such as passages or connectors for insertion into the tubes 16, 18, may correspond in shape, as outlined further in the following description. This ensures that the tubes 16, 18 are coupled with the correct hub 24a, 24b, or ports associated with a single hub.

Turning now to FIG. 5, an alternate version of a catheter 100 is provided (which again may be considered as a precursor for forming a custom cut-to length catheter). As noted above, the catheter 100 may include a balloon 112 at a distal end, which balloon may be connected along a distal end portion of a shaft 114. In this example, the shaft 114 may include an opening 114a adjacent to and proximal of a proximal end portion 112b of the balloon 112. This opening 114a is in communication with and provides access to a guidewire tube 116 extending from adjacent to opening 114a to a tip adjacent to a distal end portion 112a of the balloon 112. This allows for the insertion of a guidewire G (see FIG. 8) to achieve a “rapid-exchange” or RX configuration, but as outlined further herein, a side-by-side or coaxial arrangement could also benefit from the cut-to-length features of this disclosure.

In the case of catheter 100 having a balloon 112, which is considered optional, the shaft 114 may be in the form of a tube 118 including an inflation lumen for supplying an inflation fluid to an interior compartment of the balloon. A hub 124 may also be provided for connecting to the shaft 114, such as in a manner outlined further in the following discussion. The hub 124 may include a port 124a adapted for connecting to an indeflator (not shown) for inflating and deflating the balloon 112.

A tubular strain relief member 120 may be movably mounted on the shaft 114, such as for sliding along it. This strain relief member 120 may have a distal open end having a smaller diameter for engaging or closely surrounding the tube 118. A proximal open end of this member 120 includes a larger diameter for partially receiving the hub 124.

In use, this tubular strain relief member 120 may overlie the junction where the shaft 114 joins the hub 124. The member 120 may also be fabricated of a flexible, resilient material (such as, for example, an elastomer). When positioned over the joint thus formed, this member 120 thus serves to relieve strain and reduce the potential for kinking, such as during insertion of the catheter 100 into a body lumen.

As can be understood from FIGS. 5, 6, and 7, the shaft 114 includes one or more markings M visible to a user. The markings M may comprise external lines, notches, or other indicia, and may comprise one or more numbers. The number(s) may correspond, for example, to a distance from the marking M to a location on the catheter 100, such as the distal tip adjacent to the distal end portion 112a of the balloon (or a tip separate from the balloon). The distance(s) may be in conventionally used and understood units for catheters, such as centimeters, and may be associated with one or more lines to form a scale or ruler along the shaft 114.

When a catheter 100 of a particular length shorter than a nominal length is desired for use in performing a particular medical procedure, the user simply locates the corresponding marking M on the shaft 114. In the example shown in FIG. 6, this selected marking M corresponds to a distance of 130 centimeters (not shown to scale in the drawings), which may correspond to the length from the marking to the tip corresponding to the distal end 112a of the balloon 112 (with the full length of the catheter 100 being greater than 200 centimeters, as indicated). From experience or as a result of provided instructions, the user may also appreciate that a relatively small portion of the shaft 114 is inserted into the hub 124. Accordingly, the chosen location for the cut may be selected to achieve the desired “working” length of the shaft 114 (i.e., the portion not within the hub 124).

The shaft 114, or in particular the tube 118 forming it, may then be cut or severed at the selected marking. The separation may be achieved using a cutter or tool, or manually by way of a frangible portion of the shaft 114 (e.g., a circumferential notch) corresponding to the location of the selected marking. Once separated, the severed portion 118a of the tube 118 may then be discarded. A passage in a proximal open end of the hub 124 receives the remaining tube portion 118b, as indicated in FIG. 7, and forms a connection with it (which may be achieved using an internal connector, such as within the hub 124, as outlined herein).

Turning to FIG. 8, completion of the custom, cut-to-length version of catheter 100 may involve moving or sliding the tubular strain relief member 120 (if present) along the shaft 114 until the hub 124 is partially received in the distal open end of this member. If necessary, the strain relief member 120 may be reduced in size, such as by heating if fabricated of a heat shrink material. Prior to use, the guidewire G may also be inserted through opening 114a and passed through the catheter tip adjacent to the distal end portion 112a of balloon 112 to allow for endovascular guidance during an intervention in a “rapid-exchange” (RX) manner.

A further embodiment of a catheter 200 adapted for being cut-to-length is shown in FIGS. 9 and 9A (which catheter, again, may be considered as a precursor for forming a custom cut-to length catheter). This catheter 200 includes a shaft 214 with at least two lumens 216, 218 in a side-by-side arrangement, which lumens may be formed by separate tubes within the shaft or passages therein. The catheter 200 otherwise may be constructed substantially as noted as catheter 100 of the previous embodiment, including with a balloon 212 carried at or along a distal end of the shaft 214, and may also include markings M to facilitate cutting shaft 214 to a desired length.

The first lumen 218 in the shaft 214 may correspond to a guidewire lumen, which may be at least partially formed by a tube 218a. This tube 218a may extend distally from shaft 214 and pass through the balloon 212 to a tip 212a thereof. In such an arrangement, a guidewire G may be passed through the first lumen 216 from a proximal end to a distal end of catheter 200 to achieve an “over-the-wire” configuration.

The second lumen 216 of the shaft 214 may be adapted to supply an inflation fluid for inflating the balloon 212, if present. This lumen 216 may terminate at a proximal end 212b of the balloon 212 to supply inflation fluid to an interior compartment thereof. Additional lumens may also be provided as necessary or desired for a particular medical procedure (such as, for example, delivery of a treatment agent, such as a pharmaceutical composition, into a body lumen in which the catheter 200 is located during use).

As noted in FIG. 9A, the side-by-side lumens 216, 218 form tubes or passages for conveying material through the shaft 214 of catheter 200, and may have different cross-sectional shapes. Corresponding connectors 226, 228 for insertion into these lumens 216, 218 may be associated with a hub 224 for connecting to a proximal end of the shaft 214. For example, as shown in FIG. 9C, a first connector 226 may be provided with a first cross-sectional shape corresponding to the cross-sectional shape of the first lumen 216 (shown as being crescent-shaped, which is optional). A second connector 228 may have a second cross-sectional shape corresponding to the shape of a second lumen 218 (shown as being circular, which is also optional). The connectors 226, 228 may have any number of barbs B or other external protrusions or like structures for forming a seal on insertion into the respective lumen 216, 218.

Alternatively or additionally, the shaft 214 and the hub 224 may each be provided with one or more markings, such as in the form of surface indicia I, which form guides for guiding the coupling of these structures in the correct orientation. Specifically, these indicia I may be adapted to align when the shaft 214 is in the correct orientation with the hub 224 for coupling (e.g., aligned such that the first lumen 216 aligns with the first connector 226 for insertion and coupling thereto, and the second lumen 218 aligns with the second connector 228 for insertion and coupling thereto). The indicia I may comprise, for example, a line or arrow, and may have a particular coloration and/or writing to facilitate recognition and achieve the correct alignment during assembly of the shaft 214 and the hub 224.

It can also be understood from FIG. 9B that the lumens 216, 218 and the connectors 226, 228 need not have different cross-sectional shapes, and may have the same cross-sectional shape (e.g., circular). As indicated in FIG. 9C, the connectors 226, 228 associated with the hub 224 could also have different diameters, and the lumens 216, 218 also formed of different diameters, to facilitate proper alignment.

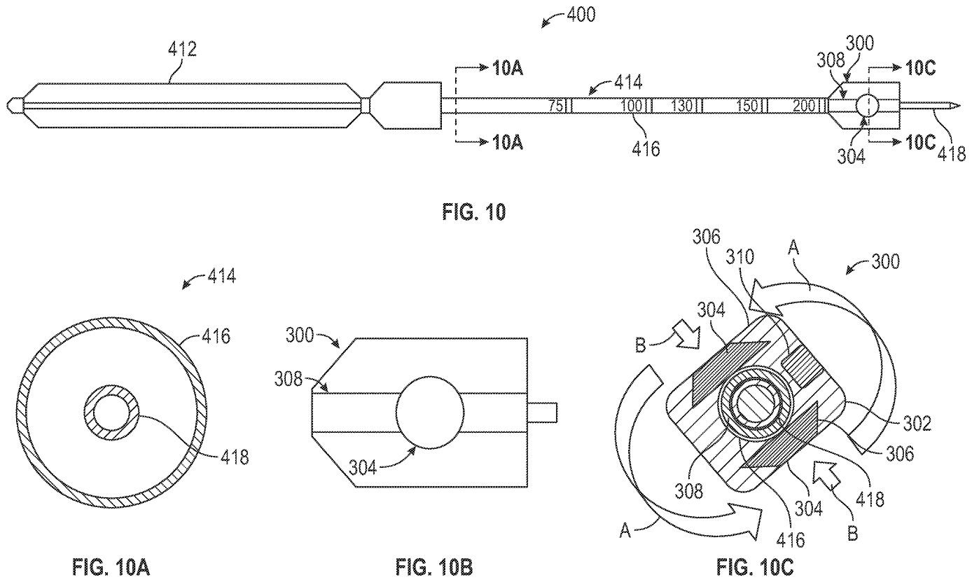

For purposes of cutting to length, a cutter 300 may be provided, as shown in FIGS. 10, 10A, 10B and 10C. The catheter 400 for use with the cutter 300 in this example may be a coaxial arrangement, as shown in FIG. 10A. As with the other disclosed embodiments, this catheter 400 may also optionally include an expandable element, such as an inflatable balloon 412, along a distal end portion.

The catheter 400 (which again may be considered as a precursor for forming a custom cut-to length catheter) comprises a shaft 414 that includes a larger diameter outer tube 416 and a smaller diameter inner tube 418. The inner or smaller tube 418 may form a guidewire lumen, which tube then passes through the interior compartment of balloon 412, and the larger tube 416 supplies inflation fluid to the balloon, such as at a proximal end 412a thereof 412.

The cutter 300 may comprise a tubular body 302 with a passage, such as for receiving the shaft 214 of the catheter 200. The body 302 may include one or more externally accessible buttons 304. These buttons 304 are adapted to move in a radial direction, such as from a home or radially outward position to a deployed or radially inward position.

To achieve the desired cutting, one or more sharpened blades 306 may also be provided within the body 302. These blade(s) 306 may be located adjacent to the radially inward end of the buttons 304. The blades 306 may be mounted for movement in the radial direction, and may also be biased to move toward shaft 214 when the buttons are pressed and away from the shaft when the buttons are released.

With the shaft 414 received in the body 302 of the cutter 300, the button(s) 304 may be depressed manually to cause the blade(s) 306 to engage the shaft 414 and, in particular, the outer tube 416. The distance of deployment may be fixed such that the blade(s) 306 only sever or cut and outer tube 416, but not inner tube 418, which may thus extend proximally of the outer tube 416 as indicated in FIG. 10. The cutting may be achieved by rotating the cutter 300 about the shaft 414 in either direction (circumferential arrows A) while pressing the buttons 304 inwardly (radial arrows B).

In one particular example, a guard 308 may be provided for guarding the coaxial inner tube 418 while the outer tube 416 is separated into portions by the cutter 300. This guard 308 may comprise a tubular body at least partially comprising a ferromagnetic or magnetic material. The guard 308 may be adapted to be inserted in an annulus between the outer tube 416 and the inner tube 418. Thus, the guard 308 may have a diameter greater than an outer diameter of the inner tube 418, but smaller than the inner diameter of the outer tube 416.

The cutter 300 and, in particular, the body 302, may be adapted to form a non-contact coupling with the guard 308. This may be achieved from external to the catheter shaft 414 by using a magnet 310 for magnetically coupling with the guard 308 when positioned in the annulus between tubes 416, 418 at the location where the cut is desired to form the custom-length catheter. Alignment of the guard 308 with the cutter 300 and, in particular, blade(s) 306 is thus assured, which protects the inner tube 418 from being inadvertently severed or damaged in the process of modifying the shaft 414 to achieve the desired length of catheter 400.

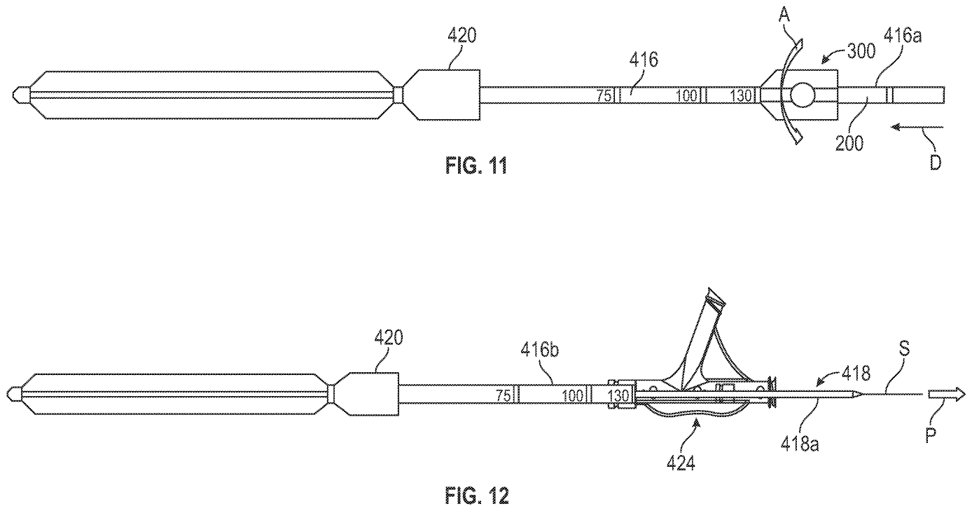

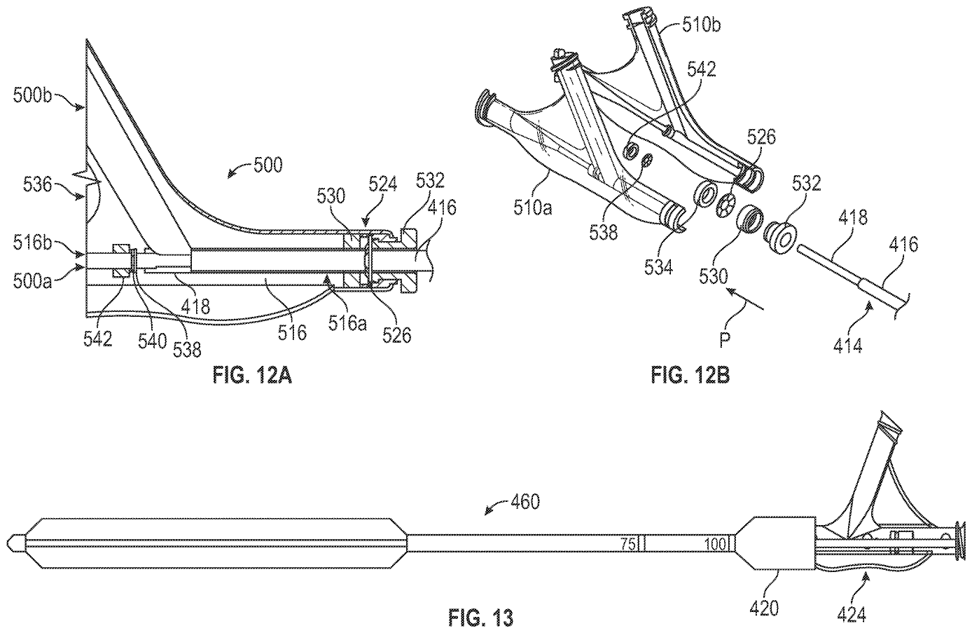

With reference to FIGS. 11, 12, and 13, the catheter 400 may be shortened to a custom length, such as by using cutter 300 to divide shaft 414 as described above or otherwise severing the material of the shaft. If using the cutter 300, it may be advanced distally along the shaft 414, as indicated by arrow D, and then used as described above to divide the outer tube 416. The cut portion 416a of the outer tube 416 may be removed to expose the inner tube 418, with the remainder of outer tube 416b having the desired length according to marking(s) M, as shown in FIG. 12.

To complete the arrangement, the hub 424 may then be passed distally over the proximally exposed portion 418a of inner tube 418 and connected in place, as outlined further in the following description. If necessary, tension may be applied to the inner tube 418, both during cutting of the outer tube 416 and installation of the hub 224, by pulling it in the proximal direction, as indicated by arrow P. This may be facilitated by attaching a draw string S to the inner tube 418, which may be passed through the hub 424 before insertion of the inner tube or attached to it thereafter. The proximal portion of the inner tube 418 uncovered by hub 424 may be cut, either prior to or following insertion so as to terminate distally of a proximal end of the hub 424.

The hub 424 may then be connected to the outer and inner tubes 416, 418, such as by using an adhesive or other bonding, or a push connector, as outlined in the following description. With the hub 424 in-stalled on the proximal end of the shaft 414, the tubular strain relief member 420 may then be moved into position covering a distal end of the hub 424, thus completing the custom, cut-to-length catheter 400, as shown in FIG. 13.

The hub 24, 124, 224, 424 may be manufactured to include the one or more connectors, such as the connectors 226, 228 previously described or other forms of connectors to achieve the desired connection with the shaft 14, 114, 214, 414. As shown in FIGS. 12A and 12B, an exemplary hub 500 (which may be used as any of the hubs noted above, and thus can be used with catheters 100, 200, or 400) may include a first connector 524 comprising a first ring 526 and a second connector 536 comprising a second ring 538 may be pre-positioned in the corresponding portions of chambers 516a, 516b of a passage 516, along with a retainer 530 and seals 534, 542.

The hub 500 may be adapted for the irremovable, one way insertion of the shaft 414 (or portions thereof) once inserted (or pushed) therein. In one example, each ring 526, 538 is provided a plurality of inwardly directed, radially extending projections or fingers for engaging the outer surface of a respective portion of the shaft 414 when passed therethrough (e.g., the outer and inner tubes 416, 418 in a coaxial arrangement, or side-by-side tubes 16, 18 in the catheter 10 shown in FIG. 3). In view of the smaller diameter of the inner tube 418, the second connector 536 may be proportionally smaller than the first connector 524 for engaging the outer, larger diameter tube 416.

As can be understood, the rings 526, 538 forming the connectors 524, 536 are fixed in the hub 500. Thus, the inwardly directed, radially projecting fingers of these rings 526, 538, engage the outer and inner tubes 416, 418, respectively, as a result of being pushed therethrough. This engagement biases the fingers proximally, which thus prevents relative distal movement of the tubes 416, 418 and essentially renders them immovable in the distal direction.

As can be appreciated, using this arrangement allows for the proximal end of the shaft 414 to be introduced into the hub 500 and mechanically captured in place. Specifically, in the arrangement shown in FIGS. 12A and 12B, the shaft 414 is passed in a proximal direction P through the collar 532, the connector 524, and the seal 534, as well as connector 536 and seal 542 in the internal passage 516.

If pre-cut prior to insertion into the hub 500, the inner tube 418 proximal end extends into the passage 516 in communication with a first port 500a of the hub 500. If not pre-cut prior to insertion into the hub 500, then the inner tube 418 extends proximally through the passage 516 beyond the proximal end of hub 424, as shown in FIG. 12, and the ex-tended portion 418a and may be cutoff. In either case, this inner tube 418 becomes irremovably captured in place within the hub 500 due to the engagement with the radially inward ends of the fingers of the ring 538 forming the second connector 536.

The outer tube 416 of the shaft 414 located within the hub 500 as a result of insertion is likewise irremovably captured in place by engagement with the radially inward ends of the fingers of the first, larger connector 524 captured in the hub 500. In this position, the opening of the inflation lumen provided by the annulus between the outer and inner tubes 416, 418 is located so as to be in communication with a second port 500b of the hub 500, which may be used for supplying inflation fluid to an associated balloon on a distal end of the shaft 414.

As a consequence of the engagement with fingers 528, 540, separation of the shaft 414 from the hub 599 by pulling in the distal direction is not possible. The shaft 414 thus remains securely mechanically attached to the hub 500 in a fully sealed arrangement. In view of the mechanical nature of the connection thus formed, it can be established without the use of adhesives or bonding, if desired.

In any of the foregoing embodiments, and unless otherwise specified, conventional materials may be used for forming the various components of the catheters 10, 100, 200, 400, the cutter 300, or the associated hubs 24a, 24b, 124, 224, 424, or 500. In order to form a secure connection between these structures, adhesives or bonding techniques (such as thermal or ultrasonic welding) could be used, and/or a mechanical connection may be used (such as the push-fit arrangement).

Summarizing, this disclosure relates to the following items:
1. A method for manufacturing a custom length catheter including a hub from a shaft including an outer tube covering an inner tube, comprising:
   removing a portion of the outer tube to uncover a portion of the inner tube; and
   connecting the outer tube or the inner tube to the hub.
2. The method of item 1, further including the step of cutting the outer tube prior to the removing step.
3. The method of item 2, further including the step of providing a guard for guarding the inner tube prior to cutting the outer tube.
4. The method of item 3, wherein the step of providing the guard comprises:
   inserting the guard into an annulus between the outer tube and the inner tube; and
   forming a non-contact coupling between the guard and a cutter external to the outer tube.
5. The method of item 3, wherein the step of providing the guard comprises inserting the guard into an annulus between the outer tube and the inner tube to a distance corresponding to a marking on the outer tube indicating a length of a remaining portion of the outer tube following the cutting and removal steps.
6. The method of any of items 1-5, wherein the connecting step comprises connecting the outer tube to a first connector of the hub.
7. The method of item 6, wherein the connecting step comprises connecting the inner tube to a second connector of the hub.
8. The method of item 6 or 7, wherein the connecting step comprises pushing the outer tube through the first connector.
9, The method of items 6 to 8, further including the step of cutting a portion of the inner tube.
10. The method of item 9, wherein the cutting step is performed prior to the passing step.
11. The method of any of items 1-10, wherein the inner tube comprises a first inner tube and further including a second inner tube removably attached to the first inner tube, and the method comprises:
   detaching the first and second inner tubes; and
   connecting at least the first inner tube to the hub.
12. The method of item 11, further including the step of connecting the second inner tube to the hub or to another hub.
13. A method for manufacturing a custom length catheter from a shaft including first and second lumens, comprising:
connecting the shaft to a hub including a first connector for insertion into either the first lumen or the second lumen.
14. The method of item 13, wherein the hub includes a second connector, and the method comprises inserting the second connector into the other of the first lumen or the second lumen.
15. The method of item 12 or item 13, further including the step of aligning a first guide on the hub with a second guide on the shaft to ensure connection in the correct orientation so that the first connector is connected to either the first lumen or the second lumen.
16. The method of item 12 or item 13, further including the step of aligning a first guide on the hub with a second guide on the shaft to ensure that the first connector is connected to the first lumen and the second connector is connected to the second lumen.
17. An apparatus for forming a custom length catheter for performing a medical procedure, the catheter having a shaft including an inner tube and an outer tube, comprising:
   a cutter adapted for cutting the outer tube; and
   a removable guard for guarding the inner tube to protect against cutting by the cutter.
18. The apparatus of item 17, wherein the cutter is adapted for forming a non-contact coupling with the guard.
19. The apparatus of item 17 or item 18, further including a magnet external to the outer tube for forming a magnetic coupling with the guard.
20. The apparatus of any of items 17-20, wherein the cutter includes at least one blade for cutting the outer tube.
21. An apparatus for forming a catheter for performing a medical procedure, comprising:
   a catheter shaft including an outer tube and an inner tube arranged in a coaxial configuration; and
   a hub including at least one connector for irremovably connecting the inner tube or the outer tube to the hub.
22. The apparatus according to item 21, wherein the at least one connector comprises a connector captured within a passage of the hub.
23. The apparatus according to item 21 or item 22, wherein the at least one connector comprises a first connector for connecting to a proximal portion of the inner tube within the hub and further including a second connector for connecting to the outer tube.
24. An apparatus for forming a catheter for performing a medical procedure, comprising:
   a catheter shaft including first and second lumens; and
   a hub including a first connector for insertion into the first lumen and a second connector for insertion into the second lumen.
25. The apparatus of item 24, wherein the first and second lumens have different cross-sectional shapes.
26. The apparatus of item 24 or item 25, further including a first guide on the hub and a second guide on the catheter, the first and second guides arranged to ensure that the first connector is connected to the first lumen and the second connector is connected to the second lumen when the catheter shaft is associated with the hub.
27. The apparatus of any of item 24 or item 26, wherein the first and second lumens each have a cross-sectional shape that is substantially the same.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are ap-propriate to perform in the disclosed invention. However, it is to be understood that the value to which such modifiers refer is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although certain inventive concepts have been described in this disclosure in conjunction with specific embodiments, many alternatives, modifications, and variations will be apparent to those skilled in the art. Accordingly, this disclosure embraces all such alternatives, modifications, and variations that fall within the spirit and scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. A method for manufacturing a custom length catheter including a hub from a shaft including an outer tube covering an inner tube, comprising:
customizing a length of the outer tube by removing a portion of the outer tube to uncover a portion of the inner tube; and
mechanically connecting the outer tube or the inner tube to the hub.

2. The method of claim 1, further including the step of cutting the outer tube prior to the removing step.

3. The method of claim 2, further including the step of providing a guard for guarding the inner tube prior to cutting the outer tube.

4. The method of claim 3, wherein the step of providing the guard comprises:
inserting the guard into an annulus between the outer tube and the inner tube; and
forming a non-contact coupling, such as a magnetic coupling, between the guard and a cutter external to the outer tube.

5. The method of claim 3, wherein the step of providing the guard comprises inserting the guard into an annulus between the outer tube and the inner tube to a distance corresponding to a marking on the outer tube indicating a length of a remaining portion of the outer tube following the cutting and removal steps.

6. The method of claim 1, wherein the connecting step comprises connecting the outer tube to a first connector of the hub.

7. The method of claim 6, wherein the connecting step comprises connecting the inner tube to a second connector of the hub.

8. The method of claim 6, wherein the connecting step comprises pushing the outer tube through the first connector.

9. The method of claim 6, further including the step of cutting a portion of the inner tube.

10. The method of claim 9, wherein the cutting step is performed after passing the inner tube through the hub.

11. The method of claim 1, wherein the inner tube comprises a first inner tube and further including a second inner tube removably attached to the first inner tube, and the method comprises:
detaching the first and second inner tubes; and
connecting at least the first inner tube to the hub.

12. The method of claim 11, further including the step of connecting the second inner tube to the hub or to another hub.

13. A method for manufacturing a custom length catheter from a shaft including first and second lumens, comprising:
connecting the shaft to a hub including a first connector for insertion into either the first lumen or the second lumen.

14. The method of claim 13, wherein the hub includes a second connector, and the method comprises inserting the second connector into the other of the first lumen or the second lumen.

15. The method of claim 13, further including the step of aligning a first guide on the hub with a second guide on the shaft to ensure connection in the correct orientation so that the first connector is connected to either the first lumen or the second lumen.

16. The method of claim 13, further including the step of aligning a first guide on the hub with a second guide on the shaft to ensure that the first connector is connected to the first lumen and the second connector is connected to the second lumen.

17. An apparatus for forming a custom length catheter for performing a medical procedure, the catheter having a shaft including an inner tube and an outer tube, comprising:
a cutter adapted for cutting the outer tube; and
a removable guard for guarding the inner tube to protect against cutting by the cutter.

18. The apparatus of claim 17, wherein the cutter is adapted for forming a non-contact coupling with the guard.

19. The apparatus of claim 17, further including a magnet external to the outer tube for forming a magnetic coupling with the guard.

20. The apparatus of claim 17, wherein the cutter includes at least one blade for cutting the outer tube.

21-30. (canceled)
